(19) United States
(12) Patent Application Publication
(10) Pub. No.: US 2005/0074489 A1
(43) Pub. Date: Apr. 7, 2005

(54) Effervescent and effervescent-dispersion compositions for medicaments and methods of use thereof

(75) Inventors: Gilbert Rene Gonzales, New York, NY (US); Nicholas L. Gonzales, New York, NY (US)

Correspondence Address: WOOD, HERRON & EVANS, LLP, 2700 CAREW TOWER, 441 VINE STREET, CINCINNATI, OH, 45202, US

(73) Assignee: Pediamed Pharmaceuticals, Inc.

(21) Appl. No.: 10/676,408

(22) Filed: Oct. 1, 2003

(51) Int. Cl. A61K 9/46

(52) U.S. Cl. 424/466

(57) ABSTRACT

Pharmaceutical compositions comprising one or more medicaments in a pharmaceutically acceptable effervescent formulation. The effervescent formulation includes a first gas-dispersing component and a second gas-generating effervescent component, wherein at least one first gas is released from the first gas-dispersing component and at least one second gas is generated and evolved from the second gas-generating effervescent component, upon contact with a minimal amount of water. The formulation is placed in an aqueous vehicle wherein the formulation effervesces gases causes penetration, dispersion and distribution of the medicaments in the vehicle. The vehicle, which may be any ordinary food or beverage chosen by the patient, is then ingested by the patient for delivery of a dosage of the medicaments.

### BACKGROUND OF THE INVENTION

1. Field of the Invention

The present invention generally relates to the delivery of one or more medicaments to a patient, and in particular, to delivering the medicaments to the patient in an effervescent composition.

2. Description of the Prior Art

The oral administration of medicaments to both pediatric and adult patient populations can often be a challenge. Particularly, patients are often reluctant to swallow pills, tablets, capsules, or other solid dosage medicament formulations, especially when the act of swallowing is problematic for that individual. For example, global hystericus and choking due to pharyngeal and esophageal motility problems, renders it painful to swallow and often results in aversion to swallowing the formulation. In addition, patients with pharyngitis and/or a markedly swollen or an otherwise severely irritated pharynx, such as due to a bacterial infection, often makes it difficult and/or impossible for the patient to swallow a solid medicament formulation. Patients may also be reluctant to ingest a medicament formulation due to its size, shape, and taste, psychological aversion to the act of ingestion, and/or personal choice not to swallow the formulation. However, patients under a medication regimen and/or in need of the therapeutic active ingredient in the formulation must self-administer, or be administered, the dose. Thus, there is a need to provide a better method of administering a therapeutically effective dose of a medicament.

To enhance ingestion and/or absorption of active pharmaceutical ingredients, pharmaceutical formulations containing effervescent components have been proposed. For example, U.S. Pat. No. 5,178,878 discloses pharmaceutical dosage formulations including microparticles having pharmaceutical ingredients contained therein and effervescent disintegration agents to allow the solid formulation, such as a tablet, to dissolve in the mouth and release the microparticles to enhance swallowing thereof by the patient. This system was prepared to effectively mask ill-tasting pharmaceutical ingredients.

U.S. Pat. No. 5,223,264 discloses the same effervescent dosage formulations disclosed in U.S. Pat. No. 5,178,878, noted above, but directed towards pediatric administration. Particularly, the invention is directed towards providing vitamin and mineral supplements (not active ingredients in microparticles) to a child via the effervescent dosage formulation.

U.S. Pat. No. 4,687,662 discloses effervescent compositions in the form of tablets or powders comprising a therapeutic agent, a granulating agent, and a microparticulate effervescent component in an effervescent system which rapidly dissolves in water to yield a solution containing a completely dissolved therapeutic agent.

Similarly, U.S. Patent Publication No. 2002/0051752 discloses a pharmaceutical formulation in the form of a fast-dissolving tablet comprising an active ingredient and an effervescent combination of sodium glycine carbonate and an acid capable of reacting rapidly with sodium glycine carbonate to release carbon dioxide.

U.S. Patent Publication No. 2002/0076439 discloses a pharmaceutical composition comprising orally administrable dosage forms, such as a tablet or other ingestable dosage forms, which effervesces in the stomach to enhance absorption of otherwise poorly bioavailable pharmaceutical ingredients.

U.S. Patent Publication No. 2001/0006677 similarly discloses an effervescent polymeric film drug delivery system having extrudable water soluble or swellable binder, an active ingredient, and an effervescent couple prepared in a solid pharmaceutical dosage formulations adapted for direct oral or buccal administration.

While these proposed formulations and methods of delivering pharmaceutically active ingredients and other medicaments to a patient enhance delivery of the ingredient, they are conventional pharmaceutical forms of pills, tablets, and the like requiring the patient to directly, orally administer and/or swallow the formulation to deliver the active ingredient to the patient. Thus, such formulations do not address the problems associated with patients who are averted to, or otherwise have difficulty with, swallowing the same. Accordingly, there is a need to deliver a medicament to a patient in a convenient and effective manner. There is also a need to improve patient compliance with ingestion of a medicament. There is also a need to deliver the medicament in a formulation suitable for administration to patients who are otherwise averted to and/or not able to swallow. There is yet a further need to provide a method of delivering pharmaceutical medicaments to patients in an inexpensive, patient-friendly manner, while addressing the drawbacks and weaknesses associated with traditional methods of administering medicaments.

### SUMMARY OF THE INVENTION

The invention addresses weaknesses of the prior art methods for delivery of a medicament by providing pharmaceutical compositions including one or more medicaments in pharmaceutically acceptable effervescent formulations. The effervescent formulations further include a gas dispersing component and a gas-generating effervescent component. The formulation is placed in an aqueous vehicle, such as an aqueous food or beverage, containing a minimal amount of water such as at least about 0.1 ml of water and may be stirred or agitated. The vehicle may be selected by the caregiver or chosen by the patient, or it may simply be the saliva and/or other water-containing fluid in the patient's mouth upon direct ingestion. Upon contact with the water, the dispersing component releases at least one first gas, and the gas-generating component reacts to produce at least one second gas, both gases of which are released into the vehicle. The first and second gases may be the same or different. As the formulation breaks down in the vehicle, the medicament is also released. The released first gas disperses the effervescence of the second gas to enhance distribution and dispersion of the medicament within the vehicle. The effervescing second gas enhances penetration of the medicament in the vehicle. The vehicle is then orally ingested by the patient to administer the medicament.

The gas-dispersing component is generally a component, reactive with a minimal amount of water to release or “erupt” one or more first gases. Thus, this component generally comprises water-soluble ingredients, such as carbohydrates, saccharides of simple sugars and sugar derivatives, high and low caloric sugars, non-sugar sweeteners, non-sweeteners, and the like. There are known procedures for producing gasified solids and water-dissolvable solids matrices. For example, inert gases such as air, carbon dioxide, nitrogen, helium, ethylene oxide, oxygen, and the like, and combinations thereof may be combined with and “entrapped” in molten water-soluble ingredients upon cooling. The gas-dispersing component should generally comprise about 5% to about 85% of the total weight of the composition.

The gas-generating effervescent component(s) are also reactive with water to generate and release one or more second gases into the vehicle. Many acidic and basic components are known to react in the presence of water to generate gas. For example, acids, such as citric acid, tartaric acid, malic acid, fumaric acid, adipic acid, succinic acids, and the like, and combinations thereof are reactive with carbonates, or a source thereof, in water to generate CO₂ gas. Suitable sources of carbonate include, without limitation, dry solid carbonate, bicarbonate, and sesqui-bicarbonate salts of metals, such as sodium, potassium, lithium, calcium, and magnesium, and ammonium carbonate and bicarbonate. Excess basic component provides advantages, including providing a basic vehicle and/or a basic oral environment, taste masking properties, and many other benefits. The gas-generating effervescent component should generally comprise about 5% to about 85% of the total weight of the composition.

The “medicament” included in the composition and provided to the patient may be any natural or synthetic pharmaceutically active ingredient, vitamin, mineral, and the like, or a combination thereof. The medicament is included in effective dosages. Generally, effective amounts should be determined in accordance with the principles of pharmacy. The composition may also include any number of known, and frequently utilized, excipients to convey desirable and/or necessary properties to the effervescent formulation.

The composition is formulated as an ingestible solid such as, but not limited to, an oral dispersible pill, a chewable pill, a buccal adhesive pill, a tablet, a capsule, a granular powder, a troche, and a dragée. The formulations may be multi-layered to optimize disintegration of the formulation, and/or dispersion of the medicament, in the vehicle. The formulations may also be of varied shape, such as a biconcave shaped tablet to improve disintegration of the formulation and dispersion of the medicament in the vehicle. The formulation should also be substantially anhydrous to increase storage stability.

Thus, the invention provides a more favorable mechanism for orally delivering a medicament to a patient, which the patient may not have otherwise ingested, in a medium more convenient and patient-friendly than previously delivered. The combination of the gas dispersing and the effervescent components in the composition not only disperses and distributes the medicament in a patient-chosen and/or patient-acceptable vehicle, but also may provide for increased medicament solubility in the vehicle. For example, once the effervescent formulation breaks down in the vehicle to release effervesce the first and second gases, the resulting pressure and/or pH of the vehicle produced by the gases may enhance medicament solubility in the vehicle.

The invention also addresses the weaknesses of previously provided formulations. Particularly, ingesting a liquid or soft, semi-solid vehicle of patient choice should overcome aversions to swallowing associated with a sore, swollen, and/or problematic oral cavities, including the throat and/or pharynx. In addition, such patients may ingest liquid or semi-solid vehicles without additional oral irritation thereby overcoming psychological aversion to swallowing. In this manner, vehicles, which the patient chooses and readily consumes, should enhance the likelihood of ingestion and, therefore, compliance with a medication regimen.

These and other benefits and advantages of the invention will be further appreciated in light of the detailed description of exemplary embodiments below.

### DETAILED DESCRIPTION OF EXEMPLARY EMBODIMENTS

The invention will be further appreciated in light of the following definitions:

The term “effervescence” generally means the escape of a gas from a liquid or mixture (Hawley's Chemical Dictionary, pp. 432, 2001). Thus, the term “effervescent formulation”, as used herein, is intended to generally refer to a composition or mixture of components that evolve one or more gases, under proper conditions, such as upon contact with water.

The term “aqueous vehicle”, as used herein, is intended to refer to a medium or a carrier, such as a foodstuff, containing at least a minimal amount of water. Thus, the aqueous vehicle may be an oligohydrous vehicle containing a small amount of water, or it may be a vehicle having an abundance of water contained therein.

The term “foodstuff”, as used herein, is intended to refer to any safe, consumable liquid, semi-solid, or solid substance. Thus, a foodstuff would include any beverage and any food, which may be consumed by mammals of all classes and ages.

The term “aqueous-dissolvable” as used herein with reference to the “solid matrix”, is intended to refer to the matrix partially or fully dissolving in water or an aqueous medium.

The invention provides pharmaceutical compositions including one or more medicaments in effervescent formulations. In one embodiment of the invention, the effervescent formulation includes a gas-dispersing component, a gas-generating effervescent component, and a medicament to be delivered to the patient. The formulation is placed in an aqueous vehicle wherein the gas-dispersing component releases a first gas and the gas-generating effervescent component generates and releases a second gas. Contact with the water content of the vehicle effervesces the gases, which generally serve to penetrate and disperse the medicament within the vehicle.

The gas-dispersing component contains at least one first gas and is reactive with water, or a vehicle containing water, to release the at least one first gas into the water or vehicle. In one embodiment of the invention, the gas-dispersing component is an aqueous dissolvable solid matrix having one or more first gases contained therein. Suitable gas-dispersing components may be produced by dispersing the gas within a liquid, molten sugar, or other suitably dispersing liquid or medium, and then solidifying the dispersing medium to form a bubble, which contains or “entraps” the gas therein. The resulting gas-dispersing component is generally referred to as a “solid foam”. Suitable media for the aqueous dissolvable solid matrix includes, without limitation, carbohydrates, mono-saccharides, di-saccharides, poly-saccharides of simple sugars, sugar derivatives, and the like. Examples of suitable materials for forming the gas-dispersing component include, without limitation, high caloric sugars such as sucrose, lactose, glucose, d-glucose, I-glucose, maltose, dextrose, fructose, fructosan, gentiobiose, cellobiose, panose, malto-triose, malto-tetrose, arabinose, mannose, d-mannose, galactose, d-galactose, d-glyceraldehyde, amylose, allose, altose, talose, gulose, idose, ribose, erythrose, threose, lyxose, xylose, d-xylose, rhamnose, invert sugar, corn sugar, inositol, glycerol, glycogen, pectin, agar, sorbitol, mannitol and combinations thereof; low caloric sugars, such as sucralose, polyols, tagarose, trehalose, xylitol, dextrans, dextrins, dextrates, polysorbates, maltodextrin, xylitol, amylase, amylopectin, ribose, β-maltose, fucose, sialic acid (neuraminic acid), N-acetylgalactosamine, N-acetylglucosamine, sedoheptulose, ribulose, xylulose and combinations thereof; non-sugar sweeteners, such as acesulfane potassium, aspartame, neotame, saccharin, stevioside and combinations thereof; non-sweeteners, such as alitame, cyclamate, dihydrchalcones (DHCs), glycyrrhizin, thaumatin, gelatin, glycerin, triacetin, trehalose, alginates, gellan gum, cellulose, microcrystalline cellulose, xanthan gum, cellulose acetate phthalate, hydropropylcellulose, hydropropylmethylcellulose, ethylcellulose, methylcellulose, L-HPC (low-substituted hydroxypropyl cellulose), carrageenan, croscarmellose, povidone, crospovidone, starch, sodium starch glycolate, glucan, Adjumer® (polyidi[carboxylatophenoxyl[phosphazene), Pleuran (glycan), Pluronic L 121 (Poloxamer401), glyceraldehydes, dihydroxyacetone and combinations thereof; and combination carriers/floss/menstruum, such as without being limited to, directly compressed dried honey (Hony-TAB®), lactose and aspartame, lactose and cellulose, microcrystalline cellulose and carrageenan, microcrystalline cellulose and guar gum, microcrystalline cellulose and sodium carboxymethylcellulose, microcrystalline cellulose and lactose, and a sugar and starch combination.

The gases included in the gas-dispersing component should be pharmaceutically acceptable, as they may or may not be ingested with the formulation. For example, inert gases including, without limitation, carbon dioxide, nitrogen, air, helium, ethylene oxide, oxygen, and combinations thereof, are suitable. Suitable gas-dispersing components and methods of making same are described in U.S. Pat. No. 3,012,893, which disclosure is incorporated herein by reference in its entirety. Suitable gasified solids that are stable at room temperature and soluble in minimal amounts of water, or in nearly anhydrous or oligohydrous vehicles, are also described in U.S. Pat. Nos. 3,985,910; 3,985,909; 4,001,457; and 6,364,521, which disclosures are incorporated herein by reference in their entireties. These patents also describe the formulation and manufacture of gasified solid matrices, including gasified sugars.

The amount of the gas-dispersing component(s) in the effervescent formulations may vary. In general, the amount may range from about 5% to about 85% by weight of the final composition. In one embodiment of the invention, the amount ranges from about 15% and about 70% by weight. In another embodiment, the amount ranges from between about 20% to about 50% by weight of the total composition.

The effectiveness of the gas-dispersing component to disperse the medicament, and/or disperse the effervescing components, is generally related to the degree of “pop” caused by the abrupt release of gas. As disclosed in U.S. Pat. No. 4,837,039, which disclosure is incorporated herein by reference in its entirety, the quantity and intensity of each “pop” is generally dependent upon the size of the bubble, the pressure of the gas contained in the bubble, the surface tension of the bubble, and the degree of solubility of the ingredients of the solid matrices in water or an aqueous vehicle. For example, the intensity of the release of gas depends upon the relation of the pressure of the occluded gas to resistance of the film of the bubble and on the diameter of the bubble trapping the gas. More specifically, the strength and solubility of the solid dissolvable matrix affects the “popping” intensity. Its solubility is generally dependent upon the particular composition. For example, when comparing gas-trapped bubbles of gasified sorbitol with a gasified sucrose-glucose-lactose sugar mixture having a weight ratio of about 55:15:30, both bubbles having the same diameter, the sugar mixture generally produces a popping of greater intensity than that produced by sorbitol alone. Larger bubbles, which are in a meta-stable state by virtue of their internal pressure being greater than the strength of the film of the bubble surrounding the gas, provide an “explosion” when the film is dissolved or otherwise broken down. The size of the bubble generated in a mass of molten candy, using conventional formulating machinery and methods, typically depends on the viscosity of the mass of the sugar (dispersing medium) and the stirring device (type and velocity) used in preparing the dissolvable solid matrix formulation. Similarly, the quantity of “poppings” or the quantity of minute amounts of “eruptions” or “explosions” during the release of gas are also dependent upon the viscosity of the mass of the dispersing medium and the stirring device used to entrap the gas and/or crystalize the medium.

The gas-generating effervescent component includes compounds that evolve gas. These compounds should be capable of reacting upon exposure of one or both of the reactants to water, such as the water contained in aqueous fluids or other aqueous vehicles, to produce and/or evolve the gas. In one embodiment, the gas-generating effervescent component includes an acidic component and a basic component. In this instance, the acidic and basic components react, upon exposure to water, with one another to produce at least one second gas. For example, the reaction between a soluble acid, or source thereof, with an carbonate, or source thereof such as an alkaline metal carbonate, generally evolves CO₂ gas. More particularly, when such a gas-generating effervescent component combination is placed in a minimal amount of water, or water-containing vehicle such as saliva, CO₂ gas is generally produced and bubbles out of the water or aqueous vehicle.

The acidic component may be an acid or source thereof and should be safe for consumption. Suitable acids include, without limitation, food acids, acid anhydrides and acid salts. Examples of food acids include citric acid, tartaric acid, malic acid, fumaric acid, adipic acid, succinic acids, and the like. Anhydrides of the above-described acids may also be used as anhydrides generally degrade in the presence of water to generate the reactive acid. Examples of suitable acid salts include, without limitation, sodium dihydrogen phosphate, disodium dihydrogen pyrophosphate, acid citrate salts and sodium acid sulfite, Acid salts generally disassociate in water, or in the water content of the aqueous vehicle, to provide the reactive acid species. The overall solubility of the acid, or source thereof, in water will vary is appreciated by those skilled in the art. The effectiveness of the acid in generating the gas, and the amount of gas generated, is generally dependent on water solubility of the acid form in the effervescent formulation.

Similarly, the basic component may be a carbonate or source thereof and should be safe for consumption. Suitable carbonate sources include, without limitation, dry solid carbonate, bicarbonate, and sesqui-bicarbonate salts of metals, such as sodium, potassium, lithium, calcium, and magnesium. Examples of suitable carbonates include, without limitation, sodium carbonate, sodium bicarbonate, sodium sesquicarbonate, potassium carbonate, potassium bicarbonate, potassium sesquicarbonate, magnesium carbonate, sodium glycine carbonate, L-lysine carbonate, arginine carbonate, and amorphous calcium carbonate. Ammonium carbonate and ammonium bicarbonate are also suitable carbonates. In addition, any combination of the above sources of carbonate may be used as the basic component in the formulation.

The gas-generating effervescent component(s) are not limited to components reactive to form only carbon dioxide gas. Pharmaceutically safe reactants that evolve oxygen, nitrogen, helium, ethylene oxide, or other inert gases are also considered within the scope of the invention. For example, peroxides such as hydrogen peroxide, sodium peroxide and the like are capable of releasing useful oxygen gas. The combination of horse-radish with hydrogen peroxidase for example, or a vegetable peroxidase, is known to evolve oxygen gas. In addition, the gas-generating effervescent component(s) are not limited to mutually reactive components, such as the acidic and basic components described above, but may include safe, compounds, reactive with water to release a gas. Use of safe gas-generating effervescent component(s) and gases generated therefrom is particularly important in formulations including medicaments designed for pediatric administration.

Where the gas-generating effervescent component includes two mutually reactive components, however, it is advantageous for both components to react completely. To completely react, the reactive species in the acidic and basic component(s) should be equal in molar ratio. For example, where the acid is diprotic, then either twice the amount of a mono-reactive carbonate base or a molar equivalent of a di-reactive base should be used for complete neutralization and maximum evolution of CO₂ gas. However, the invention is not so limited, and the amount of either the acidic component or basic component may exceed the amount of its counter-part component. This excess may be useful to enhance taste and/or performance of the effervescent formulation. Where desirable properties are conveyed, the un-reacted overage is acceptable where the excess amounts are pharmaceutically safe.

Advantages may be gained where the basic component is present in a molar equivalent greater than the acidic component. In one embodiment, the composition includes the acidic component to basic component ratio in a range from about 1:1 to about 1:10 to provide complete consumption of the acidic component in the gas-generating reaction by reaction with the basic component in the aqueous vehicle. In another embodiment, the composition includes the acidic component to basic component in a ratio in the range from about 1:2 to 1:7. In yet another embodiment, the ratio is in the range from about 1:5 to about 1:6. Where the reactive equivalents of the acidic and basic components are equal, the above-disclosed “equivalence” ratios may be used as weight ratios for the amounts of the acidic component(s) relative the basic component(s). For example, an acid with two proton equivalents may be used with sodium carbonate (Na₂CO₃) in weights (mg or gm amounts) within the ranges above.

Excess basic component also provides additional benefits. For example, the excess may serve to neutralize any acidic components in the vehicle, thereby reducing potential gastrointestinal upset after ingestion of the vehicle. Moreover, basic components generally neutralize saliva (normal saliva has a pH of about 6.5 to about 6.9) and may even provide a basic pH in the oral environment, thereby enhancing absorption of lipophilic medicaments through the oral mucosa. As such, it is herein contemplated that the amounts of acidic and basic components may be adjusted so as to enhance absorption of the medicament(s) into the body, such as through the oral mucosal tissue, sublingual tissue, buccal tissue, or the gastrointestinal tract.

The amounts of the gas-generating effervescent component in the effervescent formulations may vary. In general, the amount may range from about 5% to about 85% by weight of the final composition. In one embodiment of the invention, the amount ranges from about 15% and about 70% by weight. In another embodiment, the amount ranges from between about 20% to about 50% by weight of the total composition.

The gas(es) contained in the gas-dispersing component(s) and the gas generated by the gas-generating effervescent component(s) may be the same or different. As different gases have different inherent properties, and particularly different gas pressures, the gases may be chosen as desired to “pop” and/or evolve from the effervescent formulation in the aqueous vehicle.

The effervescent formulations include one or more medicaments for administration to the patient. The term “medicament” as used herein, is intended to refer to any pharmaceutical ingredient, including active ingredients, vitamins, minerals, and the like. For example, the composition may include any of the following agents, many of which are well-known drugs, as the medicament:
Analgesic anti-inflammatory agents, such as acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like;
Drugs having an action on the central nervous system, for example sedative agents, hypnotic agents, anti-anxiotic agents, analgesic and anesthetic agents, such as chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivacaine, bupivacaine, etidocaine, prilocalne, benzocaine, fentanyl, nicotine, and the like;
Antihistaminic or anti-allergenic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, chlorpheniramine, cyproheptidine, and the like;
Anti-inflammatory agents including steroids, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, androgenic steroids, such as, testosterone, methyltestosterone, fluoxymesterone, estrogens such as, conjugated estrogens, esterified estrogens, estropipate, 17-beta estradiol, 17-beta estradiol valerate, equilin, mestranol, estrone, estriol, 17-beta ethinyl estradiol, and diethylstilbestrol; progestational agents such as progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17-alpha hydroxyprogesterone, dyd rogesterone, dimethisterone, ethinylestrenol, norgestrel, demegestone, promegestone, megestrol acetate, and the like;
Respiratory agents such as theophilline and beta₂-adrenergic agonists such as albuterol, terbutaline, metaproterenol, ritodrine, carbuterol, fenoterol, quinterenol, rimiterol, solmefamol, soterenol, tetroquinol, caffeine, caffeine citrate, and the like;
Sympathomimetic agents, such as dopamine, norepinephrine, phenylpropanolamine, phenylephrine, pseudoephedrine, amphetamine, propylhexedrine, arecoline, and the like;
Local anesthetics agents, such as benzocaine, procaine, dibucaine, lidocaine, and the like;
Antimicrobial agents including antibacterial agents, antifungal agents, antimycotic agents and antiviral agents; tetracyclines such as, oxytetracycline; penicillins such as ampicillin; cephalosporins such as cefalotin; aminoglycosides such as kanamycin; macrolides such as erythromycin, chloramphenicol, iodides, nitrofrantoin, nystatin, amphotericin, fradiomycin, sulfonamides, purroInitrin, clotrimazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like;
Antihypertensive agents such as clonidine, alpha-methyldopa, reserpine, syrosingopine, rescinnamine, cinnarizine, hydrazine, prazosin, ACE inhibitors, and the like;
Antihypertensive diuretics such as chlorothiazide, hydrochlorothrazide, bendoflumethazide, trichlormethiazide, furosemide, tripamide, methylclothiazide, penfluzide, hydrothiazide, spironolactone, metolazone, and the like;
Cardiotonic agents such as digitalis, ubidecarenone, dopamine, and the like;
Coronary vasodilators such as organic nitrates including, without limitation, nitroglycerine, isosorbitol dinitrate, erythritol tetranitrate, pentaerythritol tetranitrate, dipyridamole, dilazep, trapidil, trimetazidine, and the like;
Vasoconstrictors, such as dihydroergotamine, dihydroergotoxine, and the like;
Beta-blockers or antiarrhythmic agents, such as timolol pindolol, propranolol, and the like;
Calcium antagonists and other circulatory organ agents including, without limitation, aptopril, diltiazem, nifedipine, nicardipine, verapamil, bencyclane, ifenprodil tartarate, molsidomine, clonidine, prazosin, and the like;
Anti-convulsive agents such as nitrazepam, meprobamate, phenobarbitol, carbomazepine, valproic acid, oxazepine, phenyloin, and the like;
Agents for dizziness and nausea such as isoprenaline, betahistine, scopolamine, and the like;
Tranquilizing agents such as reserprine and chlorpromazine; and antianxiety benzodiazepines, such as alprazolam, chlordiazepoxide, clorazeptate, halazepam, oxazepam, prazepam, clonazepam, flurazepam, triazolam, lorazepam, diazepam, and the like;
Antipsychotic agents such as phenothiazines including, without limitation, thiopropazate, chlorpromazine, triflupromazine, mesoridazine, piperracetazine, thioridazine, acetophenazine, fluphenazine, perphenazine, trifluoperazine, and other major tranqulizers such as chlorprathixene, thiothixene, haloperidol, bromperidol, loxapine, and molindone, as well as those agents used at lower doses in the treatment of nausea, vomiting, and the like;
Muscle relaxants, such as tolperisone, baclofen, dantrolene sodium, cyclobenzaprine, and the like;
Drugs for Parkinson's disease, spasticity and acute muscle spasms, such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, and the like;
Respiratory agents and cough suppressants such as codeine, ephedrine, isoproterenol, dextromethorphan, orciprenaline, ipratropium bromide, cromglycic acid, and the like;
Non-steroidal hormones or antihormones such as corticotropin, oxytocin, vasopressin, salivary hormone, thyroid hormone, adrenal hormone, kallikrein, insulin, oxendolone, and the like;
Antitumor agents such as 5-fluorouracil and derivatives thereof, krestin, picibanil, ancitabine, cytarabine, and the like;
Enzymes such as lysozyme, urokinaze, and the like;
Herb medicines or crude extracts such as glycyrrhiza, aloe, Sikon (Lithospermi Radix), and the like;
Miotic agents such as pilocarpine, and the like;
Cholinergic agonists such as choline, acetylcholine, methacholine, carbachol, bethanechol, pilocarpine, muscarine, arecoline, and the like;
Antimuscarinic or muscarinic cholinergic blocking agents such as atropine, scopolamine, homatropine, methscopolamine, homatropine methylbromide, methantheline, cyclopentolate, tropicamide, propantheline, anisotropine, dicyclomine, eucatropine, and the like;
Mydriatic agents such as atropine, cyclopentolate, homatropine, scopolamine, tropicamide, eucatropine, hydroxyamphetamine, and the like;
Psychic energizers such as 3-(2-aminopropy)indole, 3-(2-aminobutyl)indole, and the like;
Humoral agents such as the prostaglandins, natural and synthetic, for example, PGE₁, PGE₂alpha, and PGF₂alpha., and the PGE₁ analog misoprostol.

Antispasmodic agents such as atropine, methantheline, papaverine, cinnamedrine, methscopolamine, and the like;
Antidepressive agents such as isocarboxazid, pheneizine, tranylcypromine, imipramine, amitriptyline, trimipramine, doxepin, desipramine, nortriptyline, protriptyline, amoxapine, maprotiline, trazodone, and the like;
Anti-diabetic agents such as insulin, and anticancer drugs such as, tamoxifen, methotrexate, and the like;
Anorectic drugs such as dextroamphetamine, methamphetamine, phenylpropanolamine, fenfluramine, diethylpropion, mazindol, phentermine, and the like;
Anti-allergenic agents such as antazoline, methapyrilene, chlorpheniramine, pyrilamine, pheniramine, and the like;
Decongestants such as phenylephrine, ephedrine, naphazoline, tetrahydrozoline, and the like;
Antipyretic agents such as aspirin, salicylamide, and the like;
Antimigrane agents such as dihydroergotamine, pizotyline, triptans, and the like;
Anti-malarial agents such as the 4-aminoquinolines, alphaminoquinolines, chloroquine, pyrimethamine, and the like;
Anti-ulcerative agents such as misoprostol, omeprazole, enprostil, and the like;
Peptides such as growth releasing factor, and the like;
Anti-estrogen or anti-hormone agents, such as tamoxifen or human chorionic gonadotropin, and the like; and
Antiulcer agents such as allantoin, aldioxa, alcloxa, -methylscopolamine methylsuflate, and the like.

The exemplary agents and drugs listed above may be used individually or in combination as required. Moreover, the drugs may be used either in their free-base form or, if capable of forming salts, in the form of a salt with a suitable counter ion, such as a suitable acid or base. Suitable acidic counter ions include, without limitation, organic acids such as methane sulfonic acid, toluene sulfonic acid, lactic acid, tartaric acid, fumaric acid, maleic acid, succinic acid, acetic acid and the like, and inorganic acids, such as hydrochloric acid, hydrobromic acid, hydrofluoric acid, phosphoric acid, sulfuric acid and the like. Suitable basic counter ions include, without limitation, organic bases such alkyl amines including triethylamine, and the like, and inorganic bases, such as sodium hydroxide, potassium hydroxide, lithium hydroxide, calcium hydroxide, ammonia and the like. If the agent has a carboxylic acid functional group, then derivatives of the acid, such as an ester, an anhydride or an amide, may be employed. The esters may include alkyl esters, aryl esters, aralkyl esters, and the like, and may also have functional groups capable of themselves forming salts. The counter ions themselves may also serve as a component for generating gas as an effervescent component.

The medicament may also be a nutritional ingredient such as a vitamin, mineral, and the like. The term “vitamin”, as used herein, includes, without limitation, thiamin, riboflavin, nicotinic acid, pantothenic acid, pyridoxine, biotin, folic acid, vitamin B₆, vitamin B₁₂, lipoic acid, ascorbic acid, vitamin A, vitamin D, vitamin E and vitamin K and derivatives thereof, calciferols, mecobalamin, and the like. Also included within the term “vitamin” are the coenzymes thereof, as coenzymes are generally beneficial agents for the body. Coenzymes include thiamine pyrophosphates (TPP), flavin mononucleotides (FMM), flavin adenine dinucleotides (FAD), Nicotinamide adenine dinucleotides (AND), Nicotinamide adenine dinucleotide phosphate (NADP), Coenzyme-A (CoA) pyridoxal phosphate, biocytin, tetrahydrofolic acid, coenzyme B₁₂, lipoyllysine, 1,1-cis-retinal, and 1,2,5-dihydroxycholecalciferol. The term “vitamin” also includes choline, carnitine, and alpha, beta, and gamma carotenes. Thus, a vitamin may include, for example, substances that may or may not be required in the diet. Salts of vitamins are also suitable.

The term “mineral”, as used herein, refers to inorganic substances, such as metal compounds and the like, generally required in the diet. Thus, suitable minerals include, without limitation, calcium, iron, zinc, selenium, copper, iodine, magnesium, phosphorus, chromium and the like, their salts, chelates, and other compositional forms and combinations thereof.

Other nutritional ingredients, commonly referred to as “dietary supplements”, include substances which have an appreciable nutritional effect when administered in small amounts. Suitable dietary supplements include, without limitation, ingredients such as bee pollen, bran, wheat germ, kelp, cod liver oil, ginseng, and fish oils, amino acids, proteins and mixtures thereof. It should be appreciated that dietary supplements may also incorporate vitamins and minerals.

The amount of the medicament included in the formulation will generally depend upon the particular medicament, its intended use, and patient profile. The amount is generally selected in accordance with known principles of pharmacy. Effective amounts are generally that amount or quantity of a drug or pharmaceutically active substance, which is sufficient to elicit the required or desired therapeutic response (biological response) when administered to a patient. In one embodiment, the effervescent formulation includes the medicament(s) in dosage amounts of up to about 1000 mg. In another embodiment, the effervescent formulation includes the medicament(s) in dosage amounts ranging from about 25 mg to about 100 mg. In yet another embodiment, the effervescent formulation includes the medicament(s) in dosage amounts of up to about 25 mg. Formulations having dosages of about 25 mg or less are generally therapeutically effective doses for a majority of pediatric medicaments, and generally sufficient dosages for many adult medications. Larger dosages will generally increase the size of the formulation. But this is not a disadvantage, as the formulation may be dissolved, degraded and otherwise delivered in a suitable vehicle of the patient's choice.

With reference to a vitamin or mineral, an effective amount is generally at least about 10% of the United States recommended Daily Allowance (“RDA”) of the particular ingredient for a patient. For example, an effective amount of vitamin C would include an amount of vitamin C sufficient to provide 10% or more of the RDA. Typically, where the formulation includes a vitamin or mineral, it will incorporate higher amounts, such as about 100% or more of the applicable RDA.

The effervescent dosage formulation may further include one or more additional adjuvants, which can be chosen from those known in the art. For example, adjuvants including flavors, diluents, colors, binders, filler, compaction agents, non-effervescent disintegrants, and the like, commonly referred to as excipients, may be included.

Examples of binders which can be used include acacia, tragacanth, gelatin, starch, cellulose materials such as methyl cellulose and sodium carboxy methyl cellulose, alginic acids and salts thereof, magnesium aluminum silicate, polyethylene glycol, guar gum, polysaccharide acids, bentonites, sugars, invert sugars and the like. Binders may be used in an amount up to about 60% by weight and advantageously from about 10% to about 40% by weight of the total composition.

Non-effervescent disintegrants include starches as corn starch, potato starch and modified starches thereof, sweeteners, clays, such as bentonite, micro-crystalline cellulose, alginates, gums such as agar, guar, locust bean, karaya, pecitin and tragacanth. Disintegrants may comprise up to about 20% by weight and advantageously between about 2% and about 10% by weight of the final composition. Notable, these binders and disintegrants may already be sufficiently present in other components of the formulation, such as in the gas-containing solid matrix.

Coloring agents may include titanium dioxide, and dyes suitable for food such as those known as F. D. & C. dyes and natural coloring agents such as grape skin extract, beet red powder, beta-carotene, annato, carmine, turmeric, paprika, etc. The amount of coloring used may range from about 0.1% to about 3.5% by weight of the final composition.

Flavors incorporated in the composition may be chosen from synthetic flavor oils and flavoring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple pear, peach, strawberry, raspberry, cherry, plum, pineapple, apricot and so forth. Flavors, which have been found to be particularly useful, include commercially available orange, grape, cherry and bubble gum flavors and mixtures thereof. The amount of flavoring may depend on a number of factors, including the organoleptic effect desired. Flavors may be present in an amount ranging from about 0.5% to about 3.0% by weight of the composition. Commonly accepted flavors include grape and cherry flavors, and citrus flavors such as orange. It is also appreciated that inclusion of flavoring agents can also influence the final flavor of the vehicle, furthering compliance with ingestion of the medicament.

A bioadhesive, such as a bioadhesive polymer, may be included in the formulation to increase the contact time between the dosage form and the oral mucosa, particularly where the dosage form is administered directly into the oral cavity and the vehicle is saliva. Non-limiting examples of known bioadhesives, or mucoadhesives, include carbopol (various grades), sodium carboxy methylcellulose, methylcellulose, polycarbophil (Noveon AA-1), hydroxypropyl methylcellulose, hydroxypropyl cellulose, sodium alginate, and sodium hyaluronate.

The individual components are formulated into a solid effervescent formulation for placement in an aqueous vehicle. Suitable solid formulations include, without limitation, orally dispersable pills, chewable pills, buccal adhesive pills, tablets, capsules including hard an soft-shelled gelatin capsules, granular powder, troches, and dragees. These formulations may be prepared by techniques known in the art. For example, a pill may be manufactured by well-known pill manufacturing procedures

Tablets may be manufactured by well-known tableting procedures. In common tableting processes, materials to be tableted are deposited into a cavity, and one or more punch members are then advanced into the cavity and brought into intimate contact with the materials to be pressed, whereupon compressive force is applied. The materials are thus forced into conformity with the shape of the punches and the cavity. Hundreds, and even thousands, of tablets per minute can be produced in this fashion. Various tableting methods, well known to those skilled in the art, are comprehensively discussed in Lieberman, Pharmaceutical Dosage Form: Tablets, Vol. 1, 2^{nd} Ed., pp 372-376, New York, 1989, which disclosure is incorporated herein by reference in its entirety.

Known granulation and wet-granulation methods for forming tablets may be utilized. Granulation generally includes any process of size enlargement whereby small particles are gathered together into larger, permanent aggregates to yield a free-flowing composition having a consistency suitable for tableting. Such granulated compositions may have consistency similar to that of dry sand. Granulation may be accomplished by agitation in mixing equipment or by compaction, extrusion or globulation. Granulation also includes, for example, a process where a liquid form of a material is rendered granular, or in a solid form, by combining it with a granular core material, such as a sugar particle. Such granular material may be produced, for example, by spray-drying the liquid onto the core particle. Thus, individual materials maybe granulated to lend themselves to tableting.

Lubricants are normally used in manufacture of effervescent tablets. Without the use of an effective lubricant, tableting by use of high-speed equipment may be difficult. The term “lubricant” as used herein, means a material which can reduce the friction arising at the interface of the tablet and the die wall during compression and ejection thereof. Lubricants may also serve to prevent sticking to the punch and, to a lesser extent, the die wall as well. Lubricants, suitable for the effervescent formulation, may be used in an amount of up to 1.5% by weight, and advantageously between about 0.5% and about 1.0% by weight of the total composition.

Extrinsic or intrinsic lubricants may be incorporated in the material to be tableted. A lubricant which is directly applied to the tableting tool surface in the form of a film, as by spraying onto the die cavity and/or punch surfaces, is known as an extrinsic lubricant. Although extrinsic lubricants can provide effective lubrication, their use requires complex application equipment and methods which add cost and reduce productivity. Magnesium, calcium and zinc salts of stearic acid have long been regarded as the most efficient intrinsic lubricants in common use. Concentrations of 1% or less by weight are usually effective.

Other traditional intrinsic lubricants include hydrogenated and partially hydrogenated vegetable oils, animal fats, polyethyleneglycol, polyoxyethylene monostearate, talc, light mineral oils, sodium benzoate, sodium lauryl sulphate, magnesium oxide and the like. See Leal, et al., U.S. Pat. No. 3,042,531, the disclosure of which is incorporated herein by reference in its entirety.

The effervescent formulation may be formulated to optimize exposure of one or both of the gas-dispersing component and gas-generating effervescent component to the water content of the vehicle. For example, the formulation may contain a plurality of layers including an outermost layer and a core. Any of the components, including the medicament, the gas-dispersing component, and the gas-generating effervescent component, may be included in the outermost layer or distributed as desired between the outermost layer and the core. Thus, bi-layered or multi-layered tablets, pills and the like formulations are contemplated herein. In one embodiment, the outermost layer includes at least the gas-dispersing component so that upon exposure to the aqueous vehicle, the component dissolves and “pops” or abruptly releases gas to distribute the core components, including for example the medicament and/or the gas-generating effervescent component, throughout the vehicle as they are released. In another embodiment, the outermost layer includes the gas-generating effervescent component so as to initiate effervescence in the vehicle prior to the “pops” from the gas-dispersing component.

In yet another embodiment, the effervescent formulation is varied in shape. For example, while conventional oval shapes of a tablet or round shapes of a pill exist, the formulation may be provided in a non-traditional shape so as to increase the surface area of the formulation that is exposed to the vehicle. Particularly, for oligohydrous vehicles having minimal water content, exposing a maximum surface area of the formulation will enhance the rate of effervescence and/or the rate of gas “explosion”, thereby promoting the rate of distribution of the medicament in the vehicle. Generally, patients do not prefer to wait for a lengthy period of time before ingesting the vehicle. Therefore, in one embodiment of the invention, the solid formulation, such as a tablet or pill, has a biconcave shape to increase the surface area for contact with the vehicle. Such a shape may also comprise multiple layers, as previously discussed herein, wherein one or more layers contain one or more of the medicament(s), and the gas-dispersing and gas-generating effervescent components. Optimal exposure of these components generally minimizes the time required to disperse the medicament in the vehicle by “explosions” and/or effervescence of gas.

As the gas-dispersing component(s) and gas-generating effervescent components are water activated, the formulation should be kept in a generally dry environment with little or no moisture available to be absorbed. Accordingly, the components themselves should be generally anhydrous or in a stable hydrated form as exposure to water may prematurely disintegrate the effervescent formulation. Alternatively, a dessicant such, as calcium carbonate, may be included to keep the formulation dry.

The effervescent formulation is orally administered to the patient in a variety of ways. In one way, it is initially placed in an aqueous vehicle, where it may be further stirred and/or agitated to commence effervescence of gases within the vehicle. Vehicles containing as little as about 0.1 ml of total water content is generally suitable to commence effervescence of gas(es) from the formulation. The effervescing gases promote penetration and distribution of the medicament in the vehicle. Formulations including the gas-generating effervescent components in the outermost layer begin to effervesce, subsequently followed by “explosions” and release of gas from the gas dispersing components. Formulations including both the gas-dispersing component and the gas-generating effervescent component in the outermost layer, and which simultaneously effervesce and “erupt” gas into the vehicle, will generally distribute the medicament(s) in a shorter period of time, as appreciated by those of ordinary skill in the art. In one embodiment, the effervescent formulation is placed in a vehicle containing up to about 5 ml of water. In another embodiment, the effervescent formulation is placed in a vehicle containing between about 5 ml and about 15 ml of water. In yet another embodiment, the effervescent formulation is placed in a vehicle containing at least about 15 ml of water.

Mini-effervescent tablet formulations allow the medicament(s) to be conveniently delivered in a small amount of a pure liquid or liquid solution, such as from about 0.1 ml to about 15 ml of the liquid. Small servings in a teaspoon or a tablespoon are easy to swallow and should enhance compliance in comparison with requiring the patient to ingest a large quantity of liquid, in a cup or glass, due to poor solubility of the medicament(s).

One benefit and advantage of the invention is that the vehicle may be chosen or selected by the patient. For example, the patient may choose a food or beverage that the patient enjoys, or which the patient has a strong liking. The vehicle should contain at least a small amount of water for the effervescence to initiate. Where the patient is a child, foods such as apple sauce, yogurts, cereals, juices, fruits, and the like, which children generally like and eat regularly, are suitable vehicles for delivery of the medicament. Even a teaspoon or tablespoon of water or a popular beverage, for example, containing small amounts of water, such as about 0.1 ml to about 15 ml, is generally sufficient to cause the composition to effervesce and disperse the medicament in the water or beverage for ease of swallowing by the patient. Thus, the vehicles contemplated herein include virtually every imaginable food or beverage, thereby addressing the needs of even the most pickiest and/or stubborn of patients, in terms of food preferences.

The vehicle having the medicament dispersed therein is then ingested by the patient. The vehicle may be ingested after completion of effervescence or during the effervescence of gases, and dispersion of the medicament(s). Children, in particular, are generally fascinated by the tiny “explosions” and effervescence of gas and are likely to enjoy ingesting the vehicle before conclusion of the effervescence. The vehicle should be completely ingested to ensure administration of the entire dosage amount.

Another way of administering the formulation is by having the patient ingest the formulation directly. In such a case, the patient's saliva or other oral fluid acts as the vehicle in which the effervescence occurs. In the fluids in the patient's mouth, the formulation generally begins to disintegrate commencing the production and/or evolution of gas. Thus, the amount of gas-dispersing and gas-generating effervescent components in the formulation should be effective to provide a “popping” and/or an effervescent sensation in the mouth of the patient. In other words, the patient should be able to perceive a distinct sensation of “fizzing” or bubbling and “popping” as the formulation disintegrates in the mouth. To provide this sensation, the amount of effervescent component(s) in each formulation should be provided to generate about 20 cm³ to about 60 cm³ of gas. The “fizzing” sensation substantially enhances the organoleptic effects of the formulation. A “positive” organoleptic sensation is one which is pleasant and/or enjoyable and which can be perceived readily by a normal human being.

In either manner of administration, the effervescent formulation, should contain the effervescent components in amounts effective to assist the rapid and complete disintegration of the composition in the aqueous vehicle or in the mouth of the patient. By “rapid”, it is understood that the formulation should disintegrate in water, in an aqueous vehicle, or even in a patient's mouth in less than about 10 minutes, and desirably between about 30 seconds and about 7 minutes. In one embodiment of the invention, the formulation is a tablet which dissolves in the vehicle or mouth in between about 30 seconds and about 5 minutes. In another embodiment, it dissolves and disperses in less than about 30 seconds. Disintegration time can generally be measured by observing the disintegration time of the tablet in water at about 37° C. The tablet is immersed in the vehicle without forcible agitation. The disintegration time is the time from immersion for substantially complete dispersion of the tablet as determined by visual observation. This method for measuring disintegration times is only one of the many methods for such purpose, as known by those skilled in this art.

Many excipients included in solid formulations, such as tablets for example, are generally more slowly soluble than the tablet binder. Thus, the term “complete disintegration” of the tablet, as used herein, does not require dissolution or disintegration of such other discrete inclusions. Many factors generally affect disintegration times in a vehicle. For example, increasing the hardness of a solid formulation may increase the disintegration time just as decreasing hardness may decrease disintegration time.

The invention will be further appreciated in light of the following example.

### EXAMPLE

An acetaminophen-containing effervescent-dispersion tablet is prepared in accordance with the following component amounts and by the following method.

| Ingredient | Weight percent of Final Product |
|---|---|
| Acetaminophen | 14.05 |
| PVP | 0.17 |
| Distilled Water (80 ml) | 0 |
| Citric Acid, microparticulate | 14.05 |
| Effervescent Component | |
| Sodium Bicarbonate | 47.77 |
| Simethicone | 0.14 |
| Distilled Water (5 ml) | 0 |
| Citric Acid | 14.05 |
| Sodium Carbonate | 4.78 |
| Sugar | 1.69 |

To prepare the gas-dispersing component, glucose and corn is mixed and heated to 162° C. The resulting mixture has a moisture content of about 2.5%. The mixture is placed in a Parr reactor (a thick-shelled pressure vessel) and stirred at temperature above 100° C. while maintaining its fused condition. Carbon dioxide gas under 600 pounds per square inch pressure is admitted and the mixture is agitated for about six minutes. The reactor is rapidly cooled to 25° C. and opened. The resulting product is hard and friable and contains about 4.5 ml of carbon dioxide per gram of product. This product is broken down into particles and screened through a 0.5 mm sieve mesh.

To prepare the component containing the pharmaceutical active ingredient, polyvinylpyrrolidone (PVP) is dissolved in distilled water (80 ml). The acetaminophen (powder) is added to the PVP solution and mixed. The resulting PVP-coated acetaminophen granules are warmed to about 60° C. until dry. The dried acetaminophen is then screened through an oscillating granulator equipped with a No. 60 mesh screen (U.S. standard). Microparticulate citric acid (500 microns) is blended with the screened acetaminophen. Independently, the simethicone is dispersed in the sodium bicarbonate.

To prepare the effervescent component, distilled water (5 ml) is added to and mixed with the remainder of the sodium bicarbonate. Citric acid is added, followed by sodium carbonate. The acetaminophen-citric acid blend (above), sugar, and the simethicone-sodium bicarbonate blend are then added and mixed into a uniform blend.

Using an “industrial-strength” tableting press (i.e. a Hata controlled feed system) having a maximum diameter of 8 mm for a round tablet, a charging depth of 21/64″, a maximum tableting pre-pressure of about 14,000 kg and a maximum tableting main pressure of about 14,000 kg, the above components are formed into a tablet using the following sequential process. Using a dual feed through an orifice of a tablet press hopper, a single 0.5 mm nucleus of 4.5 ml carbon dioxide per gram of product (i.e. the fused gas-dispersing component) is dropped along with about 623 mg of the effervescence-acetaminophen blended powder. Sequentially, the effervescence-acetaminophen blend component followed by the fused gas-dispersing component and more of the effervescent-acetaminophen blend is punch-pressed into a tablet. The tablet thus formed contains a core of primarily the gas-dispersing component (carbon dioxide fused to glucose) with an outer covering of the active pharmaceutical ingredient (API) and effervescence blend component. Each tablet contains approximately 100 mg of the API (i.e. acetaminophen). An alternative method may involve roll mixing the effervescence-acetaminophen blend powder onto the 0.5 mm fused dispersal unit followed by dropping the mix into a single feed tablet press hopper for tableting compression. The resulting tablet also has a core containing the gas-dispersing component surrounded with a coating of the effervescent-API blend.

The tablets formed above may be administered to a patient by placing the tablet in a small or large amount of hydrous or oligohydrous solution. For example, the tablet may be added to a tablespoon of applesauce or a glass of water or other solutions chosen and/or acceptable to a patient. Once in the solution (aqueous vehicle) effervescence and “popping” dispersion will begin to simultaneously occur throughout the solution. This effervescence and “popping” action thoroughly and more rapidly disperses and mixes the API in the solution, than previous API-containing effervescent formulations. The solution or vehicle may be manually agitated and/or mixed in a delivery vessel, such as a spoon or glass, and administered to the patient either by the patient or by persons caring for the patient.

By virtue of the foregoing, there are provided pharmaceutical compositions comprising a medicament in a pharmaceutically acceptable effervescent formulation. The formulation further includes a gas-dispersing effervescent component and a gas-generating effervescent component and is placed in an aqueous vehicle, such as an oligohydrous vehicle containing a minimal or a small amount of water therein. Upon contact with a sufficient amount of water in the vehicle, the gas-dispersing component dissolves and/or disintegrates to “erupt” or abruptly release gas therefrom. The “poppings” or “explosions” typically fascinate the consumer. The more the consumer is fascinated, and the more he/she will likely ingest the vehicle containing the medicament. This is particularly true for children. The “eruption” of gas helps to disperse the medicament throughout the vehicle, providing a uniform distribution of the medicament in a vehicle. Thus, where the patient ingests only a portion of the vehicle, the patient would still ingest a portion of the medicament. In addition, the eruption disperses the reactive components of the gas-generating effervescent component thereby dispersing the effervescent penetration effect, which may or may not have begun. When the effervescence has begun, the eruption disperses the effervescing gases throughout the vehicle. In addition, the eruption of gas also helps to disperse any remaining gasified solids in the vehicle. The resulting dispersion generally improves the mixing and effervescence of the medicament in the vehicle.

Accordingly, the present invention provides a mechanism which overcomes problems associated with patients who are otherwise reluctant to swallow a solid medicament formulation, such as a tablet, pill, or capsule, due to physiological ailments and/or irritations in the oral cavity and/or throat, or psychological reluctance to swallowing a medication. Particularly, the invention allows the patient to orally ingest and administer a therapeutic dose of one or more medicaments in a patient-approved vehicle, such as a beverage or a soft, food, so as to render it easy and convenient for the patient to ingest. Moreover, the medicaments are dispersed in an effervescing vehicle making it more pleasant visually, as well as practically, for the patient to ingest and obtain the therapeutic benefits of the medicament(s). To this end, the invention overcomes weaknesses and drawbacks associated with the swallowing of traditional medicament formulations.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is, therefore, not limited to the specific details, representative apparatus, method, and examples described. Accordingly, departures may be made from such details without departing from the scope or spirit of Applicant's general inventive concept.

1. A pharmaceutical composition comprising:
a medicament in a pharmaceutically acceptable effervescent formulation, said effervescent formulation comprising:
at least one first gas contained within an aqueous dissolvable solid matrix, and
at least two components reactive to generate a second gas upon aqueous contact.

2. The composition of claim 1 wherein the medicament is an agent chosen from an opioid analgesic agent, a non-opioid analgesic agent, an anti-inflammatory agent, an antitussive agent, an antipyretic agent, an antibiotic agent, an antimicrobial agent, a steroidal agent, an amphetamine stimulant agent, a non-amphetamine stimulant agent, a laxative agent, an anorexic agent, an antihistaminic agent, an antiasthmatic agent, an antidiuretic agent, an antiflatulant agent, an antimigraine agent, an antispasmodic agent, an antidiabetic agent, a respiratory agent, a sympathomimetic agent, an H₂ blocking agent, an antihyperlipidemic agent, an antihypercholesterol agent, a cardiotonic agent, a vasodilating agent, a vasoconstricting agent, a sedative agent, a hypnotic agent, an anticonvulsant agent, a muscle relaxing agent, an antipyschotic agent, an antianxiolitic agent, an antihyperactive agent, an antihypertensive agent, an antitumor agent, a soporific agent, a tranquilizer, a decongestant, a beta-blocker, a non-steroidal hormone, an herbal agent, an enzyme, a humoral agent, a madriatic agent, a psychic energizer, a vitamin, a mineral, and combinations thereof.

3. The composition of claim 1 wherein the matrix comprises a saccharide.

4. The composition of claim 1 wherein the matrix comprises an aqueous dissolvable material selected from the group consisting of sucrose, lactose, glucose, maltose, dextrose, fructose, fructosan, gentiobiose, cellobiose, panose, malto-triose, malto-tetrose, arabinose, mannose, galactose, amylose, allose, altose, talose, gulose, idose, ribose, erythrose, threose, lyxose, xylose, rhamnose, invert sugar, corn sugar, inositol, glycerol, glycogen, pectin, agar, sorbitol, mannitol, sucralose, polyols, tagarose, trehalose, xylitol, dextrans, dextrins, dextrates, polysorbates, maltodextrin, xylitol, amylase, amylopectin, ribose, β-maltose, fucose, sialic acid (neuraminic acid), N-acetylgalactosamine, N-acetylglucosamine, sedoheptulose, ribulose, xylulose, acesulfane potassium, aspartame, neotame, saccharin, stevioside, alitame, cyclamate, dihydrchalcones (DHCs), glycyrrhizin, thaumatin, gelatin, glycerin, triacetin, trehalose, alginates, gellan gum, guar gum, cellulose, microcrystalline cellulose, xanthan gum, cellulose acetate phthalate, low-substituted hydroxypropyl cellulose, hydropropylcellulose, hydropropylmethylcellulose, ethylcellulose, methylcellulose, carrageenan, croscarmellose, povidone, crospovidone, starch, sodium starch glycolate, glucan, Adjumer® (polyidi[carboxylatophenoxyl[phosphazene), Pleuran (glycan), Pluronic L 121 (Poloxamer 401), glyceraldehyde, dihydroxyacetone, directly compressed dried honey and combinations thereof.

5. The composition of claim 1 wherein the matrix releases the at least one first gas upon contact with an aqueous vehicle containing at least about 0.1 ml of water.

6. The composition of claim 1 wherein the at least two components reactive to generate the second gas comprises at least one acidic component and at least one basic component.

7. The composition of claim 6 wherein the acidic component is chosen from citric acid, tartaric acid, amalic acid, fumaric acid, adipic acid, lactic acid, succinic acid, disodium hydrogen phosphate, sodium dihydrogen phosphate, and combinations thereof.

8. The composition of claim 6 wherein the basic component is chosen from sodium carbonate, sodium bicarbonate, sodium sesquicarbonate, potassium carbonate, potassium bicarbonate, potassium sesquicarbonate, magnesium carbonate, sodium glycine carbonate, L-lysine carbonate, arginine carbonate, amorphous calcium carbonate, ammonium carbonate, ammonium bicarbonate and combinations thereof.

9. The composition of claim 6 wherein the acidic component to basic component equivalence ratio is in a range from about 1:1 to about 1:10.

10. The composition of claim 6 wherein the acidic component to basic component equivalence ratio is in a range from about 1:2 to about 1:7.

11. The composition of claim 1 wherein the at least two components are capable of generating the second gas upon contact with an aqueous vehicle containing at least about 0.1 ml of water.

12. The composition of claim 1 wherein the at least one first gas is an inert gas chosen from carbon dioxide, nitrogen, air, helium, ethylene oxide, oxygen, and a combination thereof.

13. The composition of claim 1 wherein the second gas is carbon dioxide.

14. The composition of claim 1 wherein the first gas is the same as the second gas.

15. The composition of claim 1 wherein the first gas is different from the second gas.

16. The composition of claim 1 in an ingestible formulation chosen from an oral dispersible pill, a chewable pill, a buccal adhesive pill, a tablet, a capsule, a granular powder, a troche, and a dragée.

17. The composition of claim 1 in an ingestible formulation comprising a plurality of layers including an outermost layer and a core, the outermost layer containing at least one of the medicament, the solid gas-containing matrix, and the second gas-generating reactive components.

18. The composition of claim 1 in a solid formulation comprising a plurality of layers including an outermost layer and a core, the core containing at least one of the medicament, the solid gas-containing matrix, and the second gas-generating reactive components.

19. A pharmaceutical composition comprising a medicament in a pharmaceutically acceptable effervescent formulation, said effervescent formulation comprising:
a first component comprising a gasified water soluble solid matrix capable of abruptly releasing at least one first gas upon contact with an aqueous vehicle, and
a second effervescent component comprising a gas-generating mixture of an acid and a base in a formulation substantially devoid of water, the mixture generating a second gas upon water contact.

20. The composition of claim 19 wherein the medicament is an agent chosen from an opioid analgesic agent, a non-opioid analgesic agent, an anti-inflammatory agent, an antitussive agent, an antipyretic agent, an antibiotic agent, an antimicrobial agent, a steroidal agent, an amphetamine stimulant agent, a non-amphetamine stimulant agent, a laxative agent, an anorexic agent, an antihistaminic agent, an antiasthmatic agent, an antidiuretic agent, an antiflatulant agent, an antimigraine agent, an antispasmodic agent, an antidiabetic agent, a respiratory agent, a sympathomimetic agent, an H₂ blocking agent, an antihyperlipidemic agent, an antihypercholesterol agent, a cardiotonic agent, a vasodilating agent, a vasoconstricting agent, a sedative agent, a hypnotic agent, an anticonvulsant agent, a muscle relaxing agent, an antipyschotic agent, an antianxiolitic agent, an antihyperactive agent, an antihypertensive agent, an antitumor agent, a soporific agent, a tranquilizer, a decongestant, a beta-blocker, a non-steroidal hormone, an herbal agent, an enzyme, a humoral agent, a madriatic agent, a psychic energizer, a vitamin, a mineral, and combinations thereof.

21. The composition of claim 19 wherein the medicament is present in the formulation in an amount of about 1000 mg or less.

22. The composition of claim 19 wherein the medicament is present in the formulation in an amount ranging from about 25 mg to about 100 mg.

23. The composition of claim 19 wherein the medicament is present in the formulation in an amount of about 25 mg or less.

24. The composition of claim 19 wherein the first gas is the same as the second gas.

25. The composition of claim 19 wherein the first gas is different from the second gas.

26. The composition of claim 19 in an ingestable formulation having a biconcave shape.

27. A method of administering a medicament to a patient, the method comprising:
providing to the patient a solid ingestable pharmaceutical composition comprising the medicament, an autodispersing first gas component, and a second gas-generating effervescent component, the components reactive with an amount of an aqueous vehicle containing at least about 0.1 ml of water to generate the first and second gases, and providing that the patient orally administers the composition in an aqueous vehicle.

28. The method of claim 27 further comprising the patient selecting the aqueous vehicle for administering the composition.

29. The method of claim 27 further comprising instructing the patient to combine the composition with an amount of the aqueous vehicle containing up to about 5 ml of water.

30. The method of claim 27 further comprising instructing the patient to combine the composition with an amount of the aqueous vehicle containing at least about 5 ml of water.

31. The method of claim 27 further comprising instructing the patient to combine the composition with an amount of an aqueous vehicle containing water in an amount ranging about 5 ml to about 15 ml.

32. The method of claim 27 further comprising combining the composition with an aqueous vehicle containing at least about 0.1 ml of water.

33. The method of claim 27 wherein the aqueous vehicle is at least one of water, saliva, and a foodstuff.

34. The method of claim 27 further comprising administering the combination of the composition and the aqueous vehicle to the patient.

35. The method of claim 27 further comprising formulating the composition into one of an oral dispersible pill, a chewable pill, a buccal adhesive pill, a tablet, a capsule, a granular powder, a troche, and a dragée prior to providing the composition to the patient.

36. The method of claim 27 wherein the patient is a child.

37. A method of enhancing patient compliance with a pharmaceutical therapy, the method comprising:
providing to a patient an ingestible medicament formulated with a first effervescent component and a second effervescent component, the first component dispersing the medicament upon contact with an aqueous vehicle, and the second component enhancing penetration of the medicament; and allowing the patient to select the aqueous vehicle for dispersion of the medicament in the aqueous vehicle.

38. The method of claim 37 wherein the patient is a child.

39. The method of claim 37 wherein the aqueous vehicle is at least one of water, saliva, and a foodstuff.

40. The method of claim 37 wherein the patient disperses the medicament in an amount of the aqueous vehicle containing at least about 0.1 ml of water.

41. A method of formulating an effervescent pharmaceutical composition, the method comprising:
formulating a biconcave multi-layered ingestible solid dosage formulation comprising (a) a medicament, (b) a dispersing gas, and (c) a substantially anhydrous effervescent penetration enhancing gas precursor, the dispersing gas comprising a solid aqueous-soluble matrix containing the gas, the penetrating gas precursor comprising an anhydrous admixture of an acid and a base capable of generating a gas upon aqueous contact, wherein at least one of (b) and (c) comprises an outer layer for initial aqueous contact.

42. The method of claim 41 wherein the pill is one of an oral dispersible pill, a chewable pill, and buccal adhesive pill.

43. The method of claim 41 wherein the aqueous-soluble matrix comprises an aqueous dissolvable material selected from the group consisting of sucrose, lactose, glucose, maltose, dextrose, fructose, fructosan, gentiobiose, cellobiose, panose, malto-triose, malto-tetrose, arabinose, mannose, galactose, amylose, allose, altose, talose, gulose, idose, ribose, erythrose, threose, lyxose, xylose, rhamnose, invert sugar, corn sugar, inositol, glycerol, glycogen, pectin, agar, sorbitol, mannitol, sucralose, polyols, tagarose, trehalose, xylitol, dextrans, dextrins, dextrates, polysorbates, maltodextrin, xylitol, amylase, amylopectin, ribose, 1-maltose, fucose, sialic acid (neuraminic acid), N-acetylgalactosamine, N-acetylglucosamine, sedoheptulose, ribulose, xylulose, acesulfane potassium, aspartame, neotame, saccharin, stevioside, alitame, cyclamate, dihydrchalcones (DHCs), glycyrrhizin, thaumatin, gelatin, glycerin, triacetin, trehalose, alginates, gellan gum, guar gum, cellulose, microcrystalline cellulose, xanthan gum, cellulose acetate phthalate, low-substituted hydroxypropyl cellulose, hydropropylcellulose, hydropropylmethylcellulose, ethylcellulose, methylcellulose, carrageenan, croscarmellose, povidone, crospovidone, starch, sodium starch glycolate, glucan, Adjumer® (polyidi[carboxylatophenoxyl[phosphazene), Pleuran (glycan), Pluronic L 121 (Poloxamer 401), glyceraldehyde, dihydroxyacetone, directly compressed dried honey and combinations thereof.

44. The method of claim 41 wherein the effervescent penetration enhancing gas precursor comprises at least one acidic component and at least one basic component having an acidic component to basic component weight ratio from about 1:1 to about 1:10.

45. The method of claim 44 wherein the acidic component is chosen from citric acid, tartaric acid, amalic acid, fumaric acid, adipic acid, lactic acid, succinic acid, disodium hydrogen phosphate, sodium dihydrogen phosphate, and combinations thereof.

46. The method of claim 44 wherein the basic component is chosen from sodium carbonate, sodium bicarbonate, sodium sesquicarbonate, potassium carbonate, potassium bicarbonate, potassium sesquicarbonate, magnesium carbonate, sodium glycine carbonate, L-lysine carbonate, arginine carbonate, amorphous calcium carbonate, ammonium carbonate, ammonium bicarbonate and combinations thereof.

47. A method of dispersing a medicament in an aqueous vehicle, the method comprising:
providing to the patient a solid ingestible pharmaceutical composition comprising the medicament, an autodispersing first gas component, and a second gas-generating effervescent component, the components to disperse the medicament in the aqueous vehicle.

48. The method of claim 47 further comprising effervescing the aqueous vehicle to enhance penetration of the medicament in the vehicle.

49. The method of claim 48 further comprising dispersing the effervescence in the aqueous vehicle.

50. The method of claim 47 wherein the aqueous vehicle is saliva in the mouth of the patient.
